(19) United States
(12) Patent Application Publication
(10) Pub. No.: US 2008/0188845 A1
(43) Pub. Date: Aug. 7, 2008

(54) Tissue fusion instrument and method to reduce the adhesion of tissue to its working surfaces

(76) Inventors: Francis T. McGreevy, Aurora, CO (US); Katherine R. Pavlovsky, Denver, CO (US)

Correspondence Address: JOHN R LEY, LLC, 5299 DTC BLVD, SUITE 610, GREENWOOD VILLAGE, CO, 80111, US

(21) Appl. No.: 11/701,858

(22) Filed: Feb. 1, 2007

(51) Int. Cl. A61B 18/04

(52) U.S. Cl. 606/29

(57) ABSTRACT

Tissue sticking is substantially reduced or eliminated altogether by smoothing a working surface of a ceramic material or material with a ceramic-like surface microstructure of a tissue fusion instrument to an Ra in the range of less than 0.15 to 0.40 microns. The ceramic material may be aluminum nitride. Reducing or eliminating tissue sticking is particularly advantageous when fusing tissue.

### CROSS REFERENCE TO RELATED APPLICATIONS

This invention is related to inventions for Apparatus and Method for Rapid Reliable Electrothermal Tissue Fusion, described in U.S. patent application Ser. No. (attorney docket 24.357) and for Apparatus and Method for Rapid and Reliable Electrothermal Tissue Fusion and Simultaneous Cutting, described in U.S. patent application Ser. No. (attorney docket 24.367), both filed concurrently herewith by the inventors hereof and assigned to the assignee of the present invention. The disclosures of these concurrently-filed U.S. patent application Ser. Nos. are incorporated herein by this reference.

### FIELD OF THE INVENTION

This invention relates to the tissue fusion or tissue fusion and simultaneous cutting, and more specifically, to a new and improved electrothermal or electrosurgical instrument and method that reduce or eliminate the extent to which tissue sticks to a working surface of the instrument during the surgical procedure. The present invention is particularly useful in sealing or fusing tissue together or for simultaneously fusing and cutting tissue due to eliminating or substantially reducing the extent to which tissue sticks to the working surfaces of the instrument. Reducing or eliminating tissue sticking significantly increases the integrity and strength of the seal created.

### BACKGROUND OF THE INVENTION

Coaptive tissue fusion or sealing involves the application of force and electrical energy to heat compressed tissue sufficiently to join together separate pieces of tissue. Tissue fusion avoids the need to manually suture or tie-off tissues or vessels during a surgical procedure.

Although the exact details of the physical chemistry involved in tissue fusion are probably not completely understood, it is believed that the heat denatures chains or strands of tissue proteins in the separate pieces of tissue and the pressure causes the denatured protein chains to reconstitute or re-nature across the interface between the tissue pieces. The reconstituted proteins chains interact and intertwine with one another to hold the previously-separate tissues pieces together.

Collagen is one type of protein chain that appears to play an important role in tissue fusion. Collagen, also known as tropocollagen, consists of three polypeptide protein chains that form a triple helix. These protein chains are grouped or tangled together to establish significant tissue structure and strength, as is observed in blood vessels and ligaments. Applying heat to the tissue to raise the temperature to about 60-70° C. causes the protein chains to become disordered, disassociated, separated and untangled from the triple helix.

Elastin is another type of protein chain that appears to play an important role in tissue fusion. Elastin a collection of polypeptide protein chains that are individually and randomly cross-linked with each other to form a fibril. Fibrils are grouped or tangled together to form an elastin fiber. Upon the application of heat to raise the temperature to about 120° C., the elastin fiber becomes disassociated into a disordered collection of individual polypeptide chains, fibrils and fibers.

The heat which causes denaturation of the collagen and elastin chains also appears to create unfavorable molecular interactions among the components of the denatured proteins, resulting in a relatively high free energy state. Atoms with the same electrostatic charge, and hydrophobic and hydrophillic regions of the protein chains, begin to interact and create repulsive forces. Force must be applied at the interface between the tissue pieces during fusion to overcome the repulsive forces and to achieve more favorable interactions of the proteins chains thereby reducing the amount of free energy. Force must also be applied at the interface to maintain the denatured protein chains in physical proximity with each other so that they will reconstitute and join the tissue pieces together.

Although this theoretical model of tissue fusion is understandable, reliable tissue fusion is difficult to achieve on a consistent basis. Fusing blood vessels is of particular interest, because vessel fusion during a surgical procedure is the primary use of tissue fusion at the present time. The integrity or strength of the seal formed is the principle concern. A poorly formed seal can fail immediately or sometime after the completion of the entire surgical procedure. If the seal fails shortly after the initial attempt to seal the vessel, the surgeon can reseal or manually close the vessel. However, if the seal has a slight amount of integrity, it may fail a few hours after experiencing the stress of pulsating fluid or blood pressure. These circumstances lead to internal bleeding, which usually only can be remedied by conducting a second surgical procedure to gain access to the failed seal and occlude it. Conducting an immediate second surgical procedure induces more trauma to the patient. Forming a seal with good structural integrity is therefore of paramount importance.

One of the factors affecting the integrity of the seal is the degree to which the tissue sticks to jaws of the instrument used to seal or fuse the vessel, after the jaws are separated to release the vessel when fusion is complete. To seal the vessel, the jaws apply compression force to the vessel while heat is applied to fuse apposite sidewalls of the vessel together at an interface. The heat from the jaws typically causes the vessel walls to adhere to working surfaces of the jaws. When the surgeon attempts to separate the jaws from the fused tissue area, the adherence of the tissue to the jaws can pull the vessel apart at the fused interface, or can tear the vessel adjacent to the fused interface. In either case, a leak or incomplete seal results from the tissue sticking to the jaws upon separation.

Even if the sticking tissue does not separate the fused interface or tear the vessel, enough tissue sticking may create enough separation force to weaken or compromise the strength of the sealed area without immediately creating a leak. The seal and surrounding vessel must be able to withstand the blood or fluid pressure that the body naturally exerts upon the vessel. Weakening the seal or the adjoining tissue increases the possibility that the seal will ultimately fail at some future time, thereby giving rise to the possibility of delayed internal bleeding. It has been estimated that the integrity of the seal of approximately 1 in 5 blood vessels sealed with prior art tissue fusion devices is compromised because the tissue sticks to the working surfaces of the jaws when the jaws were separated.

Another difficulty created by tissue sticking occurs in minimally invasive surgery. Minimally invasive or endoscopic surgery allows a surgeon to conduct a surgical procedure through only a small incision, rather than creating a large open incision which exposes the internal tissues. In a minimally invasive procedure, an elongated tubular endoscope or a cannula is inserted through the small incision and directed to the surgical site. An instrument is inserted within the endoscope or cannula and is manipulated to perform the desired surgical procedure. In the case of minimally invasive tissue fusion, the instrument compresses the tissue and delivers energy which creates the heat to fuse the tissue.

Minimally invasive surgery offers many advantages over open surgery, such as decreasing the recovery time, the post-operative pain, and the risk of infection. In general, minimally invasive surgery is preferred over open surgery if the surgical procedure permits. However, tissue sticking to a minimally invasive instrument can present a very significant problem. Tissue firmly stuck to the working surface of the instrument will prevent the surgeon from withdrawing the instrument from the surgical site. In such situations, the only option available to gain access to the instrument and separate the adhering tissue is to convert the minimally invasive procedure into an open procedure. Converting a minimally invasive procedure to an open procedure is a time-consuming process, can increase the risk to the patient, and induces more trauma.

Any time that tissue sticks to the working surface of an electrosurgical or other instrument, extra time must be devoted to separating the adhered tissue. The time devoted to separating the adhered tissue may amount to a considerable proportion of the overall time necessary to perform the surgical procedure, particularly since a significant proportion of the entire surgical procedure is consumed by sealing vessels. Tissue sticking therefore extends the time of the surgical procedure, which usually creates more trauma for the patient. The tissue sticking is a continuing distraction to the surgeon.

A severed vessel occurs in every instance of vessel fusion. The vessel may be severed before the severed end is fused, or the vessel may be fused in two locations along its length and then severed between the two fused areas. In all cases, the vessel is severed by mechanically cutting the vessel, such as with a scalpel or scissors. There are no known successful and widely used electrosurgical instruments or systems which are capable of simultaneously cutting and sealing or fusing a vessel, although simultaneous cutting and sealing offers substantial advantages in reducing the amount of time required to both fuse and cut the tissue.

### SUMMARY OF THE INVENTION

The present invention reduces or eliminates the extent to which tissue sticks to a working surface of a tissue fusion instrument, particularly an electrothermal instrument used in tissue fusion or simultaneous tissue fusion and cutting procedures. Working surfaces of the instrument have characteristics which greatly diminish or eliminate tissue sticking. As a consequence, the risks are eliminated or substantially diminished that the fused tissue will be torn open or compromised in strength when the working surfaces separate after the fusion procedure. The invention therefore contributes significantly to creating a tissue seal which has high strength and integrity, and which is not weakened or destroyed by tissue sticking when the working surfaces separate. More reliable seals are created with reduced or eliminated risks of failure. The invention reduces or eliminates the risks of having to convert a minimally invasive surgical procedure into an open procedure. When used in electrothermal procedures in general, the present invention allows the surgical procedure to be completed expeditiously and without the distraction caused by the tissue sticking to the working surfaces.

In accordance with these and other features, one aspect of the invention relates to an electrothermal or electrosurgical instrument having a working surface from which energy is applied to tissue during a surgical procedure. The working surface reduces or eliminates tissue sticking during the procedure. The working surface is formed of a ceramic material and has a smoothness defined by an Ra of no greater than 0.40 microns.

Another aspect of the present invention relates to a method of reducing sticking of tissue to a working surface of an electrothermal or electrosurgical instrument after applying energy to tissue during a tissue fusion or a tissue fusion and simultaneous cutting procedure. The method comprises using a working surface which is formed of ceramic material and has a smoothness defined by an Ra of no greater than 0.40 microns.

The ceramic material of the working surface may be aluminum nitride. Essentially no tissue sticking occurs when the working surface has a smoothness defined by an Ra of no greater than 0.15 microns. The incidence of sticking increases with the increase in Ra, but essentially the incidence of sticking is extremely limited for an Ra of no greater than 0.20 microns. For an Ra of no greater than 0.25 microns, there is a slightly increased incidence of sticking, but the incidence of sticking even for an Ra of 0.40 microns is substantially reduced compared to the incidence of sticking in the prior art.

Further subsidiary features of the invention involve transferring thermal energy from the working surface, transferring electrical current from the working surface which has been doped with an electrically conductive material, transferring compression force from the working surface to the tissue during the procedure, forming at least one rounded edge on the working surface, and forming one of the working surfaces as a convex curve.

A more complete appreciation of the present invention and its scope, and the manner in which it achieves the above and other improvements, can be obtained by reference to the following detailed description of presently preferred embodiments taken in connection with the accompanying drawings, which are briefly summarized below, and the appended claims.

### BRIEF DESCRIPTION OF THE DRAWINGS

FIG. 1 is an illustration of an tissue fusion apparatus for fusing or sealing tissue or for simultaneously fusing or sealing and cutting tissue, utilizing a tissue fusion instrument which incorporates the present invention, shown in a perspective view, and a power control device, shown in block diagram form, which delivers electrical energy to the instrument, all of which is used in a tissue fusion or a tissue fusion and simultaneous cutting procedure on a vessel, shown in perspective.

FIG. 2 is an illustration of the use of the tissue fusion apparatus shown in FIG. 1 to fuse a vessel.

FIG. 3 is an enlarged side elevational view of distal arms and jaws of the tissue fusion instrument and a cross-sectional view of the vessel shown in FIG. 1, before compressing the vessel when fusing it.

FIG. 4 is a view similar to FIG. 3, showing compression and fusion of the vessel.

FIG. 5 is a perspective view of the tissue fusion apparatus shown in FIG. 1 also showing the vessel fused as shown in FIG. 4 and the vessel which has been simultaneously fused and cut.

FIG. 6 is a perspective view of the tissue fusion instrument shown in FIGS. 1, 2 and 5, with portions broken away to show internal details of a parallel jaw movement mechanism and a handle locking and release mechanism, and with certain electrical conductors shown broken away.

FIG. 7 is an enlarged partial perspective view of top and bottom distal arms and top and bottom jaws of the instrument shown in FIGS. 1-6.

FIG. 8 is a perspective view of a bottom jaw shown in FIG. 7.

FIG. 9 is an enlarged longitudinal and vertical cross-sectional view of the bottom jaw shown in FIG. 7.

FIG. 10 is a transverse cross-sectional view of the bottom jaw shown in FIG. 9, taken substantially in the plane of line 10-10 shown in FIG. 9, and illustrating a heating element embedded in the bottom jaw.

FIG. 11 is an end elevational view of one form of the working surface of the bottom jaw shown in FIG. 8.

FIG. 12 is an end elevational view of another form of the working surface of the bottom jaw shown in FIG. 11, which is an alternative to that shown in FIG. 11.

### DETAILED DESCRIPTION

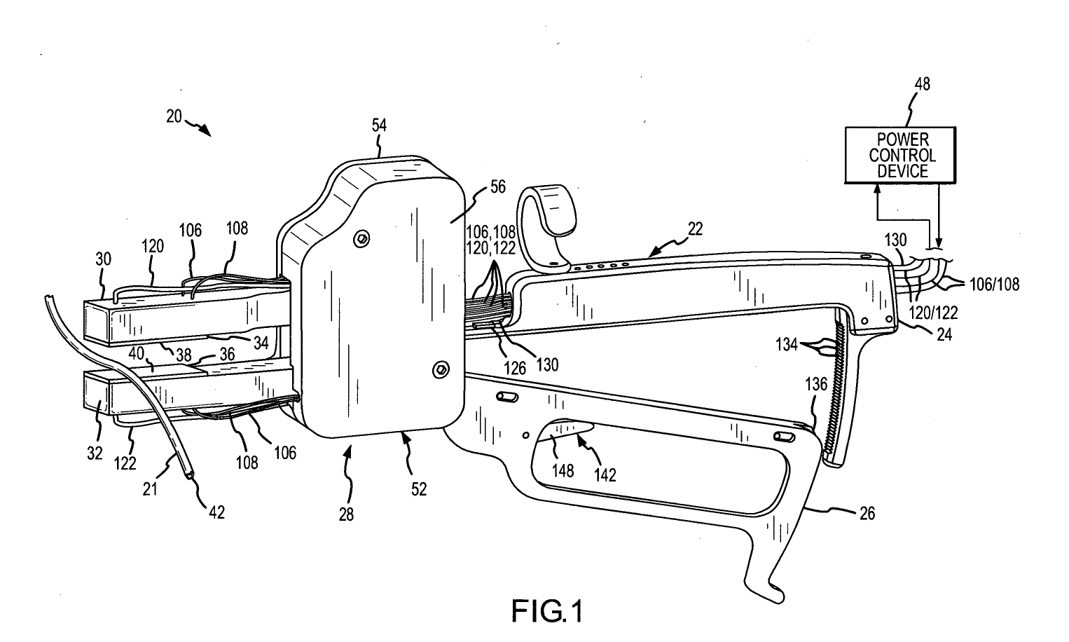

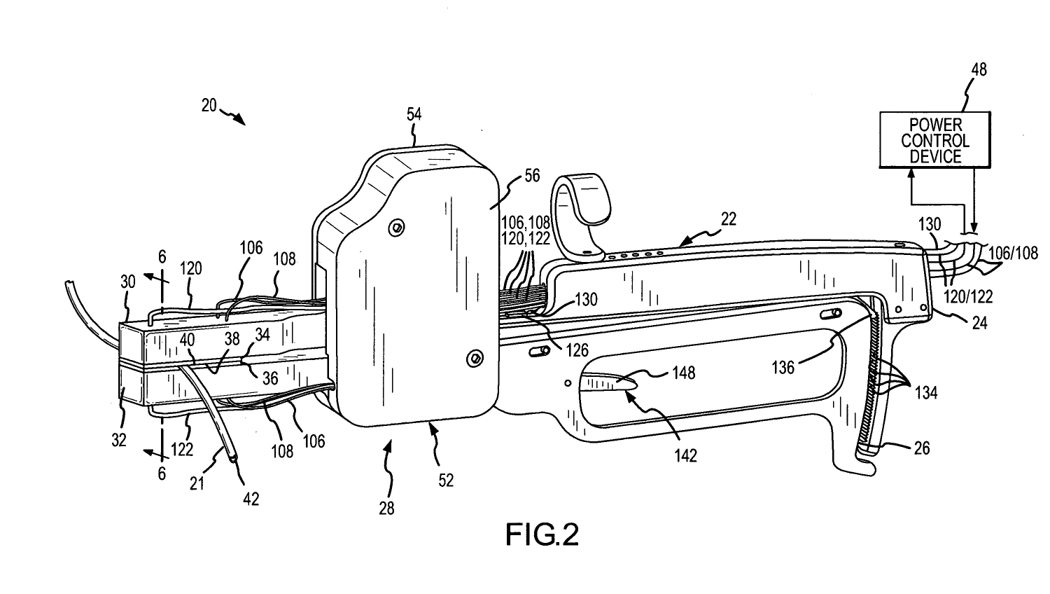

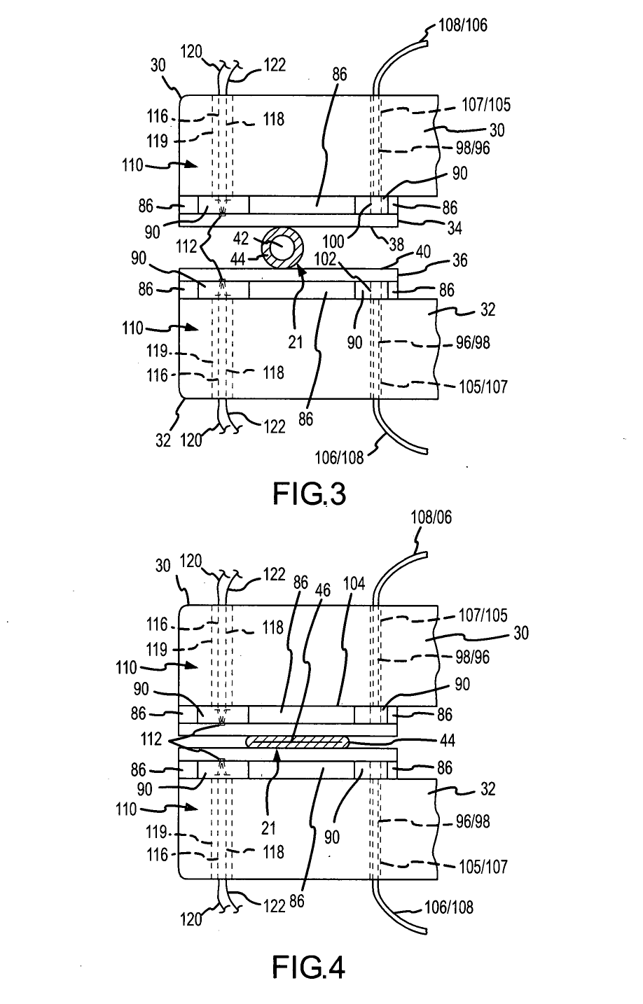

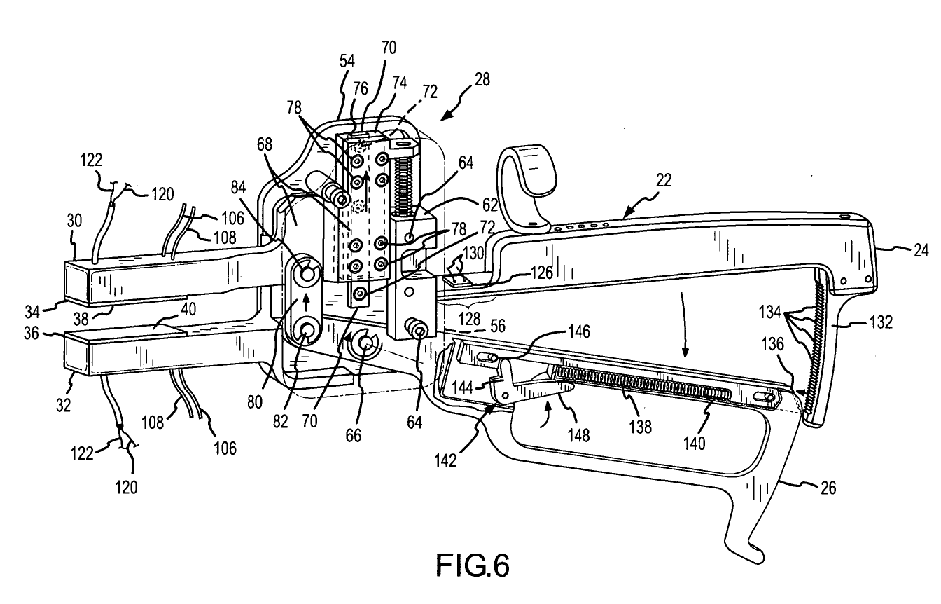

The present invention is incorporated in a tissue fusion apparatus 20 shown in FIG. 1. The tissue fusion apparatus 20 is used to fuse or seal biological tissue, such as a vessel 21, by use of a handpiece or electrothermal instrument 22, as is described more completely in the first above-referenced U.S. patent application Ser. No. (24.357), or fuse and simultaneously cut biological tissue, as is described more completely in the second above-referenced U.S. patent application Ser. No. (24.367). Proximal handles 24 and 26 of the instrument 22 are moved or squeezed together, which causes a parallel movement mechanism 28 (FIG. 6) of the instrument 22 to move distal arms 30 and 32 of the instrument 22 toward one another with parallel closing movement. Jaws 34 and 36 are attached to the distal end of the arms 30 and 32. Working surfaces 38 and 40 of the jaws 34 and 36 contact, squeeze, force and compress the vessel 21 when the jaws 38 and 40 and the distal arms 30 and 32 move toward one another, as shown in FIG. 2. Before compression, a lumen 42 within the vessel 21 is unobstructed and not occluded as shown in FIG. 3. Movement of the jaws 34 and 36 toward one another forces and compresses walls 44 of the vessel 21 into apposition with one another at a tissue interface 46 shown in FIG. 4.

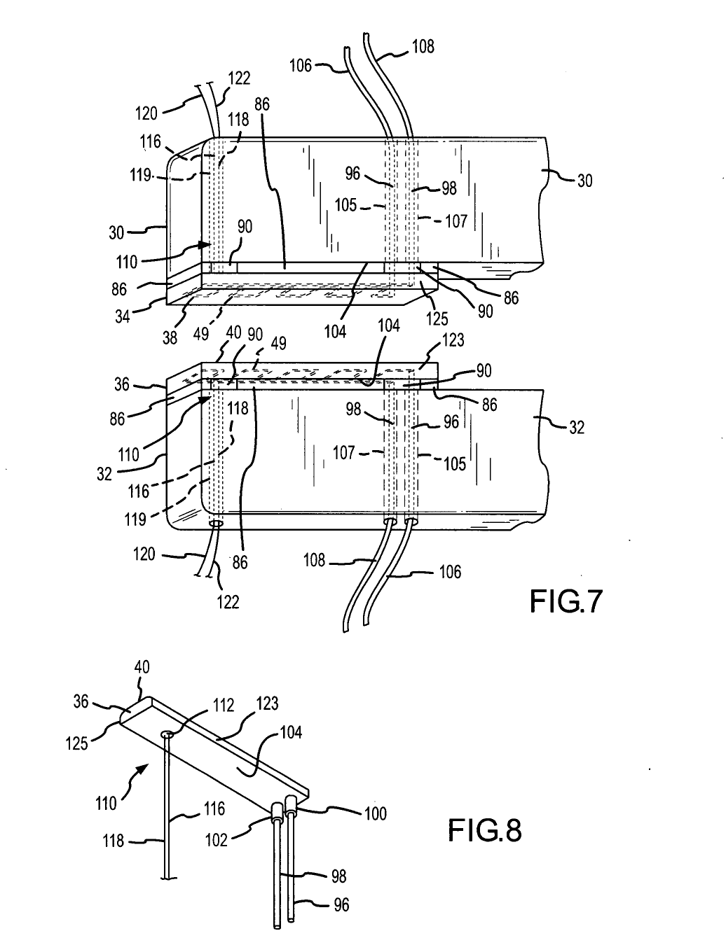

An impulse of electrical energy from a power control device 48 is delivered to a heating element 49 (FIGS. 7 and 9-12) embedded in each of the jaws 34 and 36, upon compressing together the apposite walls 44 of the vessel 21 at the tissue interface 46. The heating element 49 converts the electrical power to heat, and the heat is conducted from the working surfaces 38 and 40 to elevate the temperature of the compressed apposite vessel walls 44 at the interface 46.

To fuse the tissue, the temperature of the vessel walls 44 is elevated to a predetermined set point temperature within the range of 150° C. to 200° C., while the jaws 34 and 36 hold the apposite vessel walls 44 against one another with a force from 110 N to 150 Newtons (N). The impulse of electrical power delivered from the power control device 48 preferably has a power density of between 1000 to 1500 Watts per square inch (W/in²) (153-233 W/cm²) or greater of the working surfaces of the jaws 34 and 36, which is considerably higher than the typical power density of 115-350 W/in² (18-56 W/cm²) used in a widely used prior art RF tissue fusion device. The impulse of electrical power raises the temperature of the jaws at a preferable rate of about 150° C. to 500° C. per second. An impulse of electrical power of this magnitude is sufficient to increase the temperature of the jaws 34 and 36 to about 150° C. to 200° C. very quickly after application of the impulse.

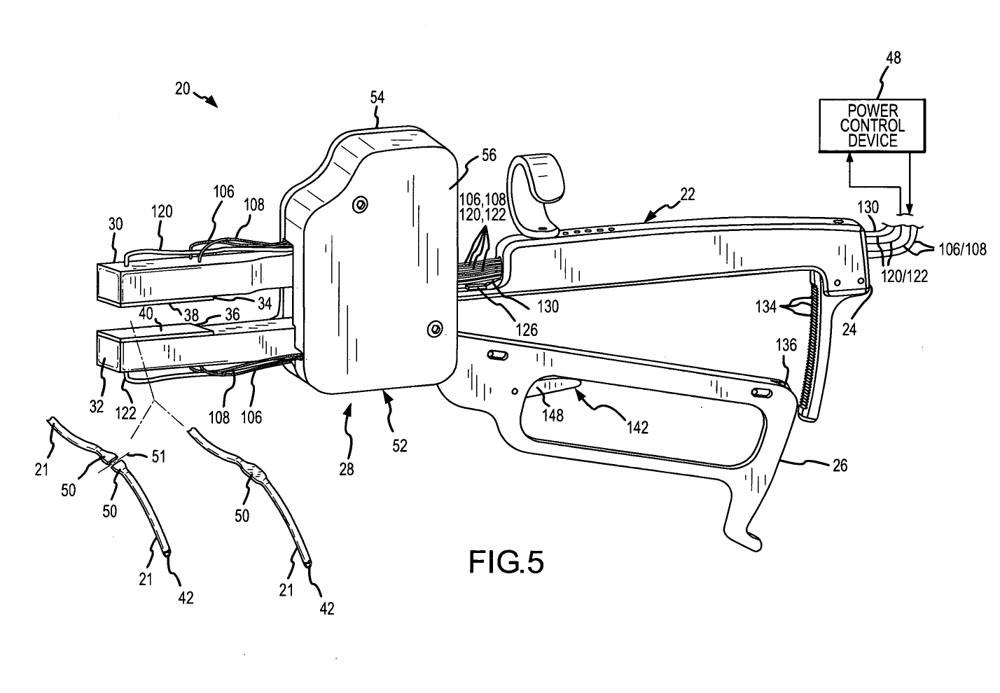

The heat denatures and disassociates the protein chains at the interface 46 of the apposite vessel walls 44 at the interface 46. The denatured protein chains immediately reconstitute or re-nature across the compressed tissue interface 46 to fuse or seal the vessel walls 44 together at a sealed area 50 (FIG. 5). The sealed area 50 occludes the lumen 42 and prevents fluid normally confined within the lumen 42 from passing from the vessel 21. The tissue fusion procedure is preferably complete in 0.5 to 2.0 seconds. Immediately after the impulse of electrical power is terminated, the handles 24 and 26 are moved away from one another, which causes the arms 30 and 32 and the attached jaws 34 and 36 to separate, releasing the vessel 21 as shown in FIG. 5.

To simultaneously cut and fuse the tissue, the temperature of the vessel walls 44 is elevated to a higher predetermined set point temperature within the range of 220° C. to 320° C., while the jaws 34 and 36 hold the apposite vessel walls 44 against one another with a force greater than 150 N. The impulse of electrical power delivered from the power control device 48 preferably has a power density of between 1500 to 2500 Watts per square inch (W/in²) (233-388 W/cm²) or greater of the working surfaces of the jaws 34 and 36. The impulse of electrical power raises the temperature of the jaws at a preferable rate of about 500° C. per second. An impulse of electrical power of this magnitude is sufficient to increase the temperature of the jaws 34 and 36 to about 220° C. to 320° C. very quickly after application of the impulse.

The thermal energy from the heat at the higher temperature is sufficient to destroy the protein chains and the other tissue components along a separation or parting line 51 while simultaneously fusing the tissue on opposite sides of the separation line 51 through the sealed area 50 (FIG. 5), in the manner described above. The sealed area 50 is substantially severed or made easily severable as a result of the heat energy delivered when the tissue is compressed. The simultaneous tissue fusing and cutting procedure is preferably complete in less than 1.5 seconds. Immediately after the impulse of electrical power is terminated, the handles 24 and 26 are moved away from one another, which causes the arms 30 and 32 and the attached to jaws 34 and 36 to separate, releasing the vessel 21 and allowing it to separate along the separation line 51 while maintaining the tissue fusion at the ends of the severed pieces of the vessel 21 along opposite sides of the separation line 51, as shown in FIG. 5.

An important aspect of the invention is the discovery that smoothing the working surfaces 38 and 40 of the jaws 34 and 36 causes the fused tissue to release from the jaws 34 and 36 without sticking when the jaws separate (FIG. 5) after completing the tissue fusion procedure or the simultaneous tissue fusing and cutting procedure, under circumstances where the working surfaces are formed of ceramic material or material which has a surface microstructure of distinct constituents like that of ceramic material, and the working surfaces have been smoothed. Tissue sticking to the working surfaces of the heated jaws is a substantial problem in prior art tissue fusion devices. If the tissue sticks to the jaws as they separate, the integrity of the fused interface at the sealed area of the vessel will be compromised by the tendency to pull the sealed vessel walls apart at the fused interface 46. Even if the fused apposite vessel walls are not separated, the separation force may weaken the walls enough to allow the natural fluid pressure within the lumen or passageway to eventually separate the vessel walls and create a leak.

Smooth working surfaces 38 and 40 formed of ceramic material allow the tissue fusion or simultaneous tissue fusion and cutting procedures to be conducted without the tissue sticking to the jaws. Thus smooth working surfaces 38 and 40 release the fused tissue from the jaws 34 and 36 without sticking when the jaws separate (FIG. 5), despite the relatively high temperature of the jaws when compressing them against the tissue. Preventing the tissue from sticking to the jaws as they separate avoids pulling the fused vessel walls apart, which could destroy or weaken the sealed area. Consequently, the fused interface of the vessel walls will have substantially all of the integrity and strength created by the fusion process, and that integrity and strength is not diminished by separation forces when the jaws separate. The smooth working surfaces 38 and 40 decrease the risk that the seal will ultimately fail. Eliminating the occurrence of tissue sticking to the jaws is a substantial improvement because sticking tissue is responsible for destroying or substantially weakening the seal in a significant proportion of those incidents where the seal failed. Eliminating the occurrence of tissue sticking to the jaws also offers a substantial convenience to surgeons, because a considerable amount of time is consumed during the surgical procedure in cleaning the jaws of adhered tissue. By avoiding the necessity to clean the jaws, a time required to perform the surgical procedure is diminished, resulting in reduced risk and trauma to the patient.

The vessel 21 exemplifies biological tissue which is sealed with the present invention, and the lumen 42 of the vessel 21 exemplifies a lumen, duct, passageway, chamber or gap or separation which is to be permanently bonded, occluded, sealed, fused or joined. The actions of bonding, occluding, sealing, fusing or joining tissues are collectively referred to herein as fusion or sealing. In addition to the vessel 21, which may be an artery or a vein, other specific examples of biological tissue which may be fused or sealed include fallopian tubes, bile ducts, tissue surrounding an aveoli or air sac in the lung, the colon or bowel, or any other tissue where surgical ligation might be performed. In most but not necessarily all of the cases where tissue fusion or sealing is performed, the purpose of sealing or fusing the tissue is to confine a fluid or other bodily substance and its associated flow within a passageway which is either defined by or closed by fusing or sealing. Therefore, in accordance with a naming convention followed in this detailed description, the walls 44 of the vessel 21 are examples of apposite pieces of biological tissue which are fused or sealed, and the lumen 42 of the vessel 21 is an example of a passageway which is permanently occluded or closed or defined by sealing the apposite walls 44 at the interface 46 of the vessel 21.

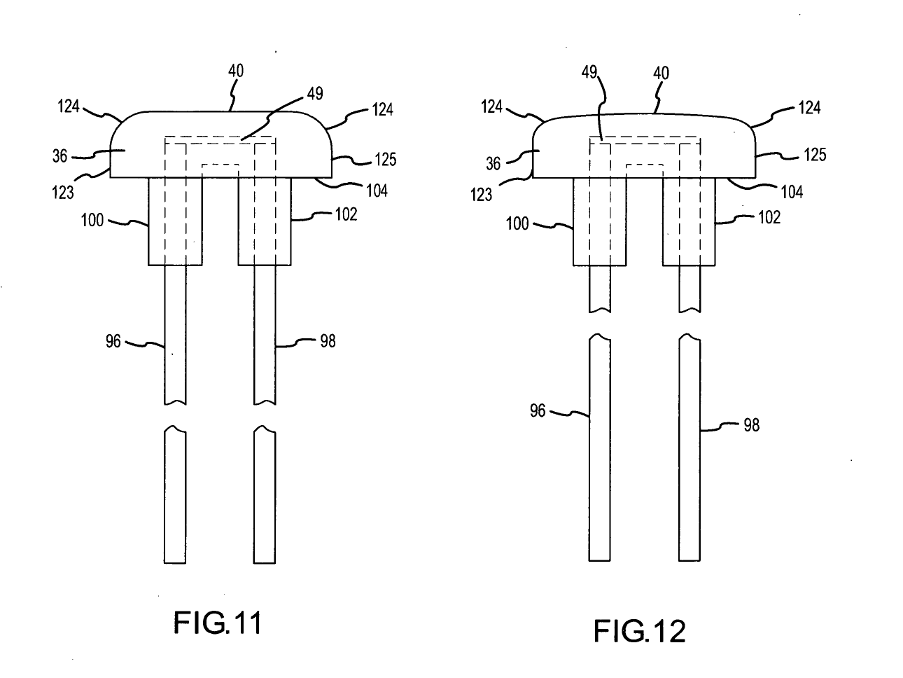

To achieve the degree of smoothness most desirable in accordance with the present invention, the working surfaces 38 and 40 and side surfaces 123 and 125 and longitudinally radiused edges 124 of the jaws 34 and 36 (FIGS. 11 and 12) should have an Ra of 0.15 microns or less. The conventional measurement of smoothness is referred to as Ra. Jaws with working surfaces which have this degree of smoothness prevent the tissue from sticking to the jaws during fusion or simultaneous cutting and fusion, despite the relatively high temperature of the jaws against the tissue.

For jaw surface smoothness in the Ra range of 0.15 to 0.40 microns, a spectrum of smoothness exists where the frequency of sticking increases in relation to decreasing smoothness, although the frequency of sticking is not in a linear relationship to decreasing smoothness. For a small increase in smoothness at the lower end of the range, a large reduction in the frequency of tissue sticking occurs. For an Ra of 0.15 microns or less, no sticking of the tissue has been observed, and the force required to separate the working surfaces from the vessel is not significantly different than if the vessel had not been present between the working surfaces. For an Ra range of 0.15 to 0.20 microns, sticking does not occur or only occurs with very minimal or virtually nonexistent frequency, and the force required to separate the working surfaces from the sealed vessel or the sealed and cut vessel is not significantly greater than the force required to separate the working surfaces from the vessel when the Ra is less than 0.15 microns. In the Ra range of 0.20 to 0.25 microns, sticking occurs with a slightly greater frequency, and the force required to separate the tissue from the working surfaces is slightly increased. In the Ra range of 0.25 to 0.40 microns, a moderate increase in the frequency of sticking occurs, and the force required to separate the tissue from the working surfaces is also moderately increased. Finally, in the Ra range of 0.40 to 0.50 microns, sticking becomes significantly more frequent and the force required to separate the tissue from the working surfaces is further increased. However, an Ra in the range of 0.40 to 0.50 microns provides less tissue sticking than with the known prior art jaws used for tissue fusion or simultaneous tissue fusion and cutting or for those jaws having an Ra of about 0.60 microns or greater.

The sticking of tissue described herein applies to that tissue upon which pressure has been applied from the working surfaces during tissue fusion. Sticking is not intended to apply to any tissue or fluid, such as blood, which remains on the working surfaces after the jaws have been separated and the sealed tissue is removed from the working surfaces. Although tissue and fluid may remain on the working surfaces after the tissue is removed, such tissue and dried fluid may easily be wiped from the working surfaces.

A conventional profilometer is used to measure the roughness of the working surfaces and to obtain the Ra values described herein. One example of such a commercially available profilometer is a Pocket Surf® portable surface roughness gauge manufactured by Mahr Federal, Inc. of Germany. Prior to measuring the roughness of the working surfaces, the profilometer was calibrated using a reference that had a known Ra. The Ra reference was certified against a known standard in accordance with ISO or ANSI standard procedures. The exemplary profilometer employed to obtain the Ra measurements described herein had an accuracy of ±0.05 microns and a resolution of 0.01 microns.

One useful ceramic material from which to form the jaws 34 and 36 is aluminum nitride. Aluminum nitride has a relatively high thermal conductivity of about 140-180 W/m°K. Aluminum nitride can also be polished to a smoothness of an Ra of 0.15 microns or less. When removed from the sintering oven after formation in a smooth mold, aluminum nitride can have an Ra as low as 0.60 microns, but not significantly lower. Working surfaces with an Ra of 0.60 microns appear smooth, but that apparent smoothness is above the acceptable range of Ra in accordance with the present invention.

Any polishing or other smoothing technique that can achieve the desired degree of smoothness of the working surfaces may be employed in accordance with the present invention. A satisfactory level of smoothness of the working surfaces of aluminum nitride jaws has been achieved by polishing the working surfaces using various grits of diamond paper or diamond pastes. Finer grades of abrasives were used in succession as the polishing proceeded toward the desired smoothness. The desired degree of smoothness was achieved by polishing the working surfaces by hand, successively using diamond grit paper with particle sizes of 6, 3, 1, 0.50, 0.25 and 0.10 microns in that order.

If the polishing is initiated with a grinding wheel or grit paper having a too coarse particle size, the working surface may be damaged and roughened to the extent that the desired smoothness can not be achieved when finer grits are used subsequently in the polishing process. When starting the polishing with too coarse of a particle size, the highest degree of smoothness (Ra of 0.15 microns or less) is difficult or impossible to achieve on aluminum nitride ceramic surfaces.

A strictly uniform smoothness across the working surfaces 38 and 40 is not required. Only those portions of the working surfaces 38 and 40 which contact the vessel 21 (FIG. 2) must be smoothed to the desired degree to obtain reduced tissue sticking. The smoothness of the radiused edges 124 and side surfaces 123 and 125 also prevents any overhanging tissue adjacent to the working surfaces 38 and 40 from sticking to the jaws 34 and 36 when the vessel 21 is sealed or simultaneously cut and sealed. To the extent that the side surfaces 123 and 125 do not touch tissue, they may not require the same degree of smoothness as the working surfaces 38 and 40 and the longitudinal radiused edges 124.

The smoothness of the working surfaces 38 and 40 of the high thermal conductivity jaws 34 and 36 contributes to creating seals of high integrity in a short amount of time. The time required for achieving a reliable seal with high integrity against leaking, or a reliable seal with simultaneous cutting, is also related to the amount of tissue squeezed between the working surfaces, the type of tissue involved and the temperature applied to the tissue. Larger vessels, thicker walled vessels or larger amounts of tissue typically require longer sealing times and/or higher temperatures. Effective seals on typical small to medium vessels of 2-3 mm diameter are achieved with electrical impulses of about 0.5 seconds duration, while seals of larger vessels in the neighborhood of 7-8 mm diameter are achieved with electrical impulses of about 2.0 seconds duration. Electrical impulses having a time duration of up to about 4.0 seconds are effective in some situations involving very large vessels and/or lower temperatures.

Because the electrical power impulse is delivered for a short period of time, the heat generated by this power does not diffuse appreciably into the surrounding walls of the vessel. As a result, the walls adjacent to the seal remain substantially unaffected by thermal energy spread. The strength and capability of the adjacent tissue is not compromised to the point where it may contribute to a failure of the seal.

Achieving consistent, reliable seals on a wide range of different sizes and types of vessels provides a significant procedural advantage over known prior art tissue sealing apparatus. Known prior art vessel sealing techniques are believed to require at least 5-12 seconds of power application before a seal is formed and the compressed vessel is released. Seals are accomplished with the present invention considerably more quickly compared to known prior art techniques. In addition, the seal created by the present invention has enhanced integrity and resistance to failure, compared to prior art seals.

Achieving tissue seals of high integrity is also accomplished by an even distribution of temperature over the working surfaces 38 and 40 of the jaws 34 and 36. Even temperature distribution is achieved by forming the jaws 34 and 36 of high thermal conductivity material, such as aluminum nitride. The high thermal conductivity material of the jaws creates a substantially uniform temperature distribution throughout the jaws 34 and 36 and on the tissue squeezed between the working surfaces 38 and 40. The tissue interface temperature is approximately equal to the temperature of the jaws 34 and 36 due to the relatively thin amount of tissue compressed between the jaws.

Achieving seals of high integrity is also accomplished by an even distribution of compression, force or pressure across the squeezed vessel walls 44 at the interface 46 (FIG. 4). The even force or pressure distribution across the tissue interface 46 is obtained by parallel movement of the working surfaces 38 and 40 toward one another when compressing the vessel 21 (FIG. 4). The parallel movement mechanism 28 causes the jaws 34 and 36 and their respective working surfaces 38 and 40 to move parallel to each other when opening and closing and compressing and releasing the vessel. The parallel movement of the jaws 34 and 36 avoids introducing shear forces on the sealed tissue interface 46 (FIG. 4) when the jaws separate. Shear forces have the effect of weakening the sealed tissue interface and diminishing the strength of the seal created.

Details of the parallel movement mechanism 28 of the instrument 22 are explained and shown mainly in conjunction with FIG. 6 but also in FIGS. 1, 2 and 5. The proximal handles 24 and 26 pivot with respect to one another in opening and closing movements. The parallel movement mechanism 28 transfers the force created by the opening and closing movements of the handles 24 and 26 into parallel opening and closing movement of the distal arms 30 and 32. The parallel opening and closing movement occurs, at a minimum, over the range of movement where the tissue is compressed between the working surfaces of the jaws during tissue fusion or simultaneous tissue cutting and fusion. The parallel movement avoids introducing adverse shear forces on the fused tissue and creates even force and pressure on the tissue when compressed.

The parallel movement mechanism 28 is enclosed within a housing 52 (FIGS. 1, 2 and 5). The housing 52 is formed by a rear wall member 54 and a front closure member 56 which includes integral top, bottom and side wall portions which enclose internal components of the the parallel movement mechanism 28. Openings are formed in the integral side wall portions of the front closure member 56 to allow the handles 24 and 26 and the arms 30 and 32 to extend into the housing 52.

The top handle 24 is integrally attached at its distal end to a block 62, and the block 62 is rigidly attached to the rear wall member 54 by pins 64. The bottom arm 32 is formed integrally with the rear wall member 54. Thus, both the top handle 24 and the bottom arm 32 are rigidly connected relative to the rear wall member 54. Thus, the top handle 24 and the bottom arm 32 do not move relative to one another or relative to the rear wall member 54 or the housing 52. Only the bottom handle 26 and the top arm 30 and jaw 34 move relative to the stationary top handle 24 and the bottom arm 32.

The bottom handle 26 is pivotally connected to the rear wall member 54 at a pivot pin 66. The bottom handle 26 pivots around the pivot pin 66. When the top and bottom handles 24 and 26 are separated or moved toward one another, only the bottom handle 26 possesses the freedom to pivot. The pivot pin 66 is located slightly proximally from the distal end of the bottom handle 26.

The top arm 30 has a flange 68 integrally attached to its proximal end. The flange 68 extends generally parallel to the rear wall member 54. As the top arm 30 moves upward and downward, the flange 68 moves upward and downward with the top arm 30 within the housing 52 between the rear wall and front closure members 54 and 56.

A rail 70 is rigidly attached to the rear wall member 54 by pins 72. The rail 70 extends perpendicularly relative to the extension of the bottom arm 32. The rail 70 projects outward from the rear wall member 54 toward the flange 68. A guide block 74 is attached to the flange 68 by pins 78. The glide block includes a center channel 76 which conforms to the cross-sectional shape of the rail 70 and which movably receives and surrounds the rail 70. The size of the center channel 76 permits a slight clearance on each the three lateral sides of the rail 70 which extend outward from the rear wall member 54. The glide block 74, flange 68 and the attached top distal arm 30 are therefore movable along a path defined by the rail 70 and relative to the rear wall member 54. dimension of the working surfaces 38 and 40 may also be planar with respect to one another (FIG. 11), but preferably one of the working surfaces has a slight convex or crowned shape (FIG. 12) while the other working surface is planar.

The parallel movement of the top and bottom arms 30 and 32 and the top and bottom jaws 34 and 36 allows the working surfaces 38 and 40 to apply and distribute force evenly across the compressed interface 46 (FIG. 4). The even force application is important to obtain even and uniform reconstitution of the denatured protein chains during fusion, resulting in enhanced strength and integrity of the sealed interface 46 (FIG. 4) and the sealed area 50 (FIG. 5). The even force application during simultaneous infusion and cutting is important to obtain even and uniform heat which separates the sealed area 50 along the separation line 51 (FIG. 5). The parallel movement of the working surfaces 38 and 40 does not impart any shearing force on the sealed area 50 (FIG. 5) as the working surfaces 38 and 40 separate from one another. Such a shearing force could compromise the integrity of the fused interface 46 (FIG. 4), apart from whether the heated and compressed vessel 21 has any tendency to stick to the working surfaces of the jaws as they separate.

The mechanical advantage resulting from closing the handles 24 and 26, transferred through the parallel movement mechanism 28, moves the arms 30 and 32 and jaws 34 and 36 to compress the vessel 21 uniformly at each point on the interface 46 of the two apposite vessel walls 44 with a force in the range of 110 N to 150 N for tissue fusion and greater than 150 N for simultaneous tissue cutting and fusion. Such a force results in compressing the pieces of tissue to a thickness of about 0.05-0.10 mm during fusion and to an essentially zero thickness during simultaneous tissue fusion and cutting. In order to achieve this range of compression, the parallel movement mechanism 28 must obtain an adequate mechanical advantage to transfer a comfortable amount of force applied on the handles 24 and 26 to the tissue. The force is related to the pressure between the working surfaces 38 and 40. The pressure is determined by the confrontational surface areas of the working surfaces 38 and 40 and the amount of force applied to the arms 30 and 32.

In a preferred embodiment, the working surfaces have a length of about 25 mm and a transverse width of about 5 mm, creating an effective confrontational surface

The heating element 49 is formed by a length of an electrically conductive resistance material which produces heat when conducting electrical current. The heating element 49 has a high thermal shock withstanding capability and a high power density conducting capability. An example of one such electrically conductive resistance material which offers these capabilities is molybdenum. The heating element 49 extends substantially over the area of the jaw 36 (FIG. 10) so that heat is produced relatively uniformly throughout the jaw. The heat from the heating element 49 is conducted substantially uniformly through the jaw 36 due to the high thermal conductivity of the ceramic material from which the jaw 36 is formed, resulting in approximately equal temperature from point to point along the working surface 40 of the jaw 36.

Electrical wires 96 and 98 connect to opposite ends of the heating element 49. Electrical current is supplied to the heating element 49 through the wires 96 and 98. The wires 96 and 98 extend through shoulders 100 and 102 which are formed on a back side 104 of the jaw from the same ceramic material as the jaw 36. The shoulders 100 and 102 surround and support the wires 96 and 98 and hold them in position as part of the jaw 36. The ceramic material of the jaw 36 is an electrical insulator, thereby assuring that the current conducted through the wires 96 and 98 flows through the heating element 49 without short-circuiting to the arms 30 and 32 of the instrument 22 (FIG. 1).

To embed the heating element 49 within the jaw 36, enough powdered ceramic material to form the working surface 40 and the outer portion of the jaw 36 is placed in a mold and sintered. Thereafter, the heating element 49 is placed on this outer partially-formed jaw portion, preferably by using conventional fluid deposition techniques such as inking. More powdered ceramic material is then placed on top of the sintered outer portion of the jaw and the heating element 49 to form the remaining inner portion of the jaw including back side 104 and the the shoulders 100 and 102. Thereafter, the powdered ceramic material which forms the inner portion of the jaw is sintered to form the ceramic inner portion of the jaw 36 while also sintering that inner portion of the jaw 36 to the previously-formed outer portion of the jaw 36, thereby completing the integral ceramic structure of the jaw.

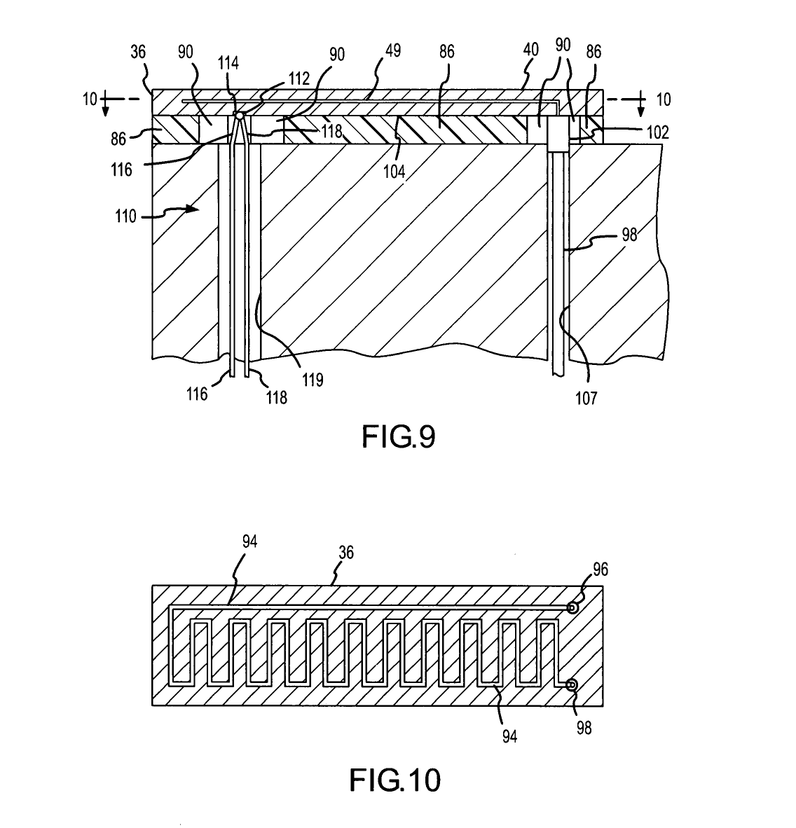

The thermocouple 110 comprises an electrical node or junction 112 of two dissimilar metal wires 116 and 118, as shown in FIG. 9. The junction of the two dissimilar wires 116 and 118 creates a conventional type JT/C thermocouple junction 112. A slight voltage is developed at the junction 112 by the inherent electrical characteristics of the two dissimilar metal wires 116 and 118, and the magnitude of that voltage varies in relationship to the temperature of the junction 112. Thus, the voltage developed at the junction 112 is related to the temperature of the junction 112. The wires 116 and 118 extend through an opening 119 formed in each arm 30 and 32 (FIGS. 3 and 4). The wires 116 and 118 may be insulated over that portion of their length which extends through the opening 119.

The voltage developed at the junction 112 is conducted through the wires 116 and 118 to conductors 120 and 122, which connect to the ends of the wires 116 and 118, respectively. The conductors 120 and 122 extend from the thermocouple 110 of each jaw 34 and 36 through the housing 52 of the parallel movement mechanism 28 and along the top handle 24 to the power control device 48, as shown in FIGS. 1 and 2. The voltage from the thermocouple 110, conducted through the conductors 120 and 122, is used by the power control device 48 as a feedback signal to control the amount of electrical current delivered through the conductors 106 and 108 and the wires 96 and 98 to the heating element 49 in the jaws 34 and 36, thereby independently regulating the temperature of the working surfaces of the jaws.

The thermocouple 110 is permanently thermally and mechanically attached to the jaw by oven brazing the junction 112 of the dissimilar metal wires 116 and 118 within a recess 114 formed into the ceramic material on the back side 104 of each jaw, as shown in FIG. 9. The attachment of the junction 112 to each jaw establishes good thermal conductivity of the junction 112 with each jaw, thereby enabling the junction 112 to respond to the temperature of the jaw. The high thermal conductivity material of each jaw distributes the heat from the heating element 49 throughout the jaw relatively rapidly. The temperature of the working surface of the jaw is typically slightly different from the temperature of the junction 112 because the junction 112 is not exactly at the working surface and slight dynamic thermal gradients exist within the jaw despite the high thermal conductivity of the jaw material. However, the

The rail 70 is oriented perpendicularly to both the top and bottom arms 30, and therefore movement of the top arm 30 maintains the same parallel angular relationship with the bottom arm 32. The rail 70 has substantial structure to withstand the torque applied to the distal end of the top arm 30 during tissue compression to maintain the same relative angular relationship of the top arm 30 with the bottom arm 32.

One end of a link 80 is pivotally connected at the distal end of the bottom handle 26 by a pivot pin 82. The other end of the link 80 is pivotally connected to the flange 68 by another pivot pin 84. Upon the clockwise (as shown in FIG. 6) pivoting movement of the bottom handle 26 relative to the top handle 24, the distal end of the bottom handle 26 transfers upward force through the link 80 to the flange 68. The flange 68 moves upward along the rail 70, and causes the connected top arm 30 to separate from the bottom arm 32. Consequently, an opening movement of the bottom handle 26 relative to the top handle 24 causes an opening separation movement of the top arm 30 relative to the bottom arm 32. A gap or separation is created between the working surfaces 38 and 40 of the top and bottom jaws 34 and 36 by the separation movement of the bottom arm 32 relative to the top arm 30.

Closing the top and bottom handles 24 and 26 moves the distal end of the bottom handle 26 downward, causing the link 80 to move the flange 68 downward along the rail 70. The top arm 30 moves downward toward the stationary bottom arm 32, thereby closing the gap between the working surfaces 38 and 40 of the top and bottom jaws 34 and 36.

The movement of the top arm 30 is restricted by the orientation of the rail 70 and the guide block 74 which is connected to the flange 68. Because the guide block 74 can only move vertically as dictated by the rail 70, the flange 68 and the integrally attached top arm 30 can only move vertically as well. The vertical motion requires the parallel angular relationship of the top and bottom jaws 34 and 36 to remain constant as the top arm 30 opens and closes relative to the bottom arm 32.

The jaws 34 and 36 are attached to the top and bottom arms 30 and 32 so that the working surfaces 38 and 40 of the jaws 34 and 36 extend parallel with one another in a longitudinal dimension extending along the arms 30 and 32. The transverse area of approximately 125 mm². The mechanical advantage must therefore be capable of producing pressure of 0.88-1.2 N per square millimeter (N/mm²) for tissue fusion and greater than 1.2 N/mm² for simultaneous tissue fusion and cutting with comfortable squeezing pressure on the handles 24 and 26. Producing a pressure of 0.88-1.2 N/mm² or greater will assure a force of 110 N-150 N or greater at each point of the compressed apposite tissue, regardless of the amount of tissue which may be compressed between the working surfaces 38 and 40. Because the pressure may vary according to the amount of tissue squeezed between the working surfaces 38 and 40, the force applied at each point to the two pieces of compressed tissue is a better measure of the effectiveness of the compression necessary to achieve good tissue fusion or simultaneous cutting and fusion than is the pressure. However, pressure must be considered to assure that an adequate amount of compression force is available.

Details of the jaws 34 and 36 are better understood by reference to FIGS. 3, 4 and 7-12. Each jaw 34 and 36 is essentially of the same structure and configuration. Each jaw 34 and 36 is preferably formed of a ceramic material with a high thermal conductivity, such as aluminum nitride. The jaws 34 and 36 are secured to the arms 30 and 32 with an adhesive, such as epoxy, which is applied in a layer 86 between the jaws 34 and 36 and the arms 30 and 32. Insulating spacers 90 are positioned near the distal and proximal ends of each of the jaws 34 and 36 between the jaws 34 and 36 and the arms 30 and 32. The adhesive layer 86 occupies the spaces between the spacers 90, the jaws 34 and 36 and the arms 30 and 32.

The heating elements 49 are embedded in the ceramic material of the jaws 34 and 36, as shown in FIGS. 7, 9 and 10. The heating element 49 in each jaw 34 and 36 is essentially the same. Similarly, both jaws 34 and 36 are essentially the same, except with respect to the possibility of one or both of the jaws having a crowned working surface (FIG. 12). Jaws having a crowned working surface are particularly useful for simultaneous tissue cutting and fusing. Because of the similarities, the heating element 49 and the jaw 36 are described in conjunction with FIGS. 8-12, with the understanding that the heating element 49 and the jaw 34 are the same.

In addition to embedding the heating element 49 within the jaw in the manner described, the heating element can also be embedded by following the described procedure but without sintering the outer portion until the inner portion has also been formed. A single sintering occurs with respect to both the outer and inner portions simultaneously to hold the heating element in place.

The wires 96 and 98 are mechanically and electrically connected to the ends of the heating element 49 by drilling holes through the shoulders 100 and 102 until the holes contact the ends of the embedded heating element 49. The wires 96 and 98 are inserted through the holes until the ends of these wires contact the ends of the heating element 49. The ends of the wires 96 and 98 and the ends of the heating element 49 are permanently connected together by brazing in an oven. The wires 96 and 98 and the shoulders 100 and 102 therefore extend from the back side 104 of the jaw 36.

When the jaws 34 and 36 are attached to the arms 30 and 32, respectively, by the adhesive layer 86, the wires 96 and 98 extend through openings 105 and 107 which are formed in each of the arms 30 and 32 to receive the wires 96 and 98, as shown in FIGS. 7 and 9. The openings 105 and 107 are sufficiently large to avoid electrical contact with the wires 96 and 98, although the wires 96 and 98 are insulated in the areas within the openings 105 and 107. Conductors 106 and 108 connect to the ends of the wires 96 and 98. The conductors 106 and 108 from each jaw 34 and 36 extend through the housing 52 of the parallel movement mechanism 28 and along the top handle 24 to the power control device 48, as shown in FIGS. 1 and 2. The power control device 48 delivers the electrical current through the conductors 106 and 108 to the heating element 49 in each jaw 34 and 36, thereby heating the jaws.

The current supplied by the power control device 48 (FIGS. 1 and 2) is regulated relative to the temperature of the working surfaces 38 and 40 of the jaws 34 and 36. The temperature of each jaw is separately measured by a thermocouple 110 associated with each jaw, shown in FIGS. 3, 4, 7 and 9. The thermocouple 110 associated with each jaw 34 and 36 is essentially the same. Therefore, only one thermocouple 110 is described in association with the jaw 36 shown in FIG. 9, since the other thermocouple is substantially identical. temperature measured by the thermocouple junction 112 is closely correlated to the temperature of the working surface of the jaw, to result in temperature measurements which closely represent the temperature of the jaw working surface. Moreover, because the tissue compressed between the jaws during tissue fusion or simultaneous tissue cutting and fusion is relatively thin, the thermal transfer to the thin tissue causes that tissue to assume a temperature which is very close to the temperature of the jaw working surfaces.

Both of the working surfaces 38 and 40 may be flat and planar as shown in FIG. 11. In such circumstances the planar working surfaces are maintained in a parallel relationship with one another by the positioning of the jaws 34 and 36 on the arms and by the parallel movement of the arms 30 and 32. Longitudinal edges 124 of the jaws 34 and 36 are rounded or radiused to avoid imparting or concentrating pressure to the vessel 21 in such a way to weaken the vessel at the edge of seal formed.

A preferred alternative to the planar configuration of the working surfaces 38 and 40 is the use of at least one crowned working surface on one of the opposing jaws. Both working surfaces could also be crowned. A crowned working surface 40 is shown in FIG. 12. The working surface 40 possesses a slight outward convex shape when viewed transversely to the longitudinal dimension of the jaw 36, as shown in FIG. 12. The crowned or convex curvature of the working surface is useful for applying more force to the vessel at the center of the working surface, while simultaneously creating a slightly graduated variation in the extent of tissue compression from the center of the working surface to the longitudinal radiused edges 124. The slight variation in compression is instrumental in achieving an optimal sealing force on the tissue squeezed between the working surfaces 38 and 40. However, in most cases, once adequate pressure is obtained, it is not necessary to achieve optimal pressure to accomplish adequate fusion, so long as the force is within the 110 N-150 N range or greater, as previously described. Furthermore, simultaneous cutting will also be achieved so long as sufficient force is ultimately applied to reduce the thickness of compressed tissue to zero at the maximum convex point of the crowned working surface(s) at the fused area.

The curvature of the crowned working surface 40 of the jaw is in the transverse direction across the working surface. The amount of curvature of the working surface 40, as shown in FIG. 12, is such that the radius of curvature of the working surface 40 in the transverse dimension is approximately 21 mm for a jaw which has a transverse width of approximately 5 mm. This radius of curvature generally causes the center of the crowned working surface to be approximately 0.1 mm higher than the working surfaces near the longitudinal edges 124 of the jaw 36, before those longitudinal edges 124 are radiused.

The amount of force transferred from the working surfaces 38 and 40 of the jaws 34 and 36 to the vessel 21 is measured by a conventional strain gauge 126 attached to a section 128 of the top proximal handle 24, shown in FIG. 6. The section 128 of the top handle 24 has a reduced cross-sectional area. The strain gauge 126 is attached to extend longitudinally along the reduced cross-sectional area section 128. Attached in this manner, the strain gauge 126 measures the amount of deflection of the section 128 created by the force resulting from squeezing the handles 24 and 26 together. The extent of deflection of the section 128 is accurately correlated to the amount of force applied from the distal arms 30 and 32 to the tissue squeezed between the working surfaces 38 and 40 (FIG. 4). The signals from the strain gauge 126 are conducted through two conductors, collectively referenced 130, to the power control device 48, where those force-related signals are used to indicate when adequate force is imparted to the compressed tissue and to control the delivery of the electrical power impulse by the power control device 48.

A handle locking and release mechanism 131 is connected to the proximal ends of the handles 24 and 26, as shown mainly in FIG. 6, and also in FIGS. 1, 2 and 5. The handle locking and release mechanism 131 includes a curved extension 132 with ratchet teeth 134 that extend downward from the proximal end of the top handle 24. The bottom handle 26 includes a ratchet pawl 136 that extends rearward from the proximal end of the bottom handle 26. The ratchet pawl 136 is connected to a rod 138 which extends longitudinally within the interior of the bottom handle 26. A spring 140 is connected between a proximal end of the rod 138 and a shoulder 141 of the bottom handle 26. The spring 140 is compressed and normally biases the rod 138 in the rearward direction. The normal rearward bias from the spring 140 on the rod 138 extends the ratchet pawl 136 rearward from the proximal end of the bottom handle 26.

When the handles 24 and 26 are squeezed together, the ratchet pawl 136 slides by and engages the individual ratchet teeth 134 in succession, until the handles 24 and 26 reach a squeezed-together position where the desired amount of force is applied on the compressed vessel 21. The handles can not separate or open because the ratchet pawl 136 is engaged with the ratchet teeth 134, thereby allowing the working surfaces 38 and 40 to maintain force on the compressed vessel 21 during fusion or simultaneous fusion and cutting. The handle locking and release mechanism 131 allows an adequate and substantial amount of force or pressure is maintained on the vessel 21 during the procedure without requiring the surgeon to continually squeeze the handles 24 and 26. The handle locking and release mechanism 131 also prevents the force or pressure on the compressed vessel from substantially decreasing during the procedure. The interaction of the ratchet pawl 136 with the ratchet teeth 134 prevents the handles 24 and 26 from moving apart from their squeezed-together position, until the ratchet pawl 136 is separated from the ratchet teeth 134.

The handle locking and release mechanism 131 includes a trigger 142 which, when squeezed, separates the ratchet pawl 136 from the ratchet teeth 134 and thereby allows the handles 24 and 26 to open with respect to one another. The trigger 142 includes a contact arm 144 which contacts and interacts with a shoulder 146 at the distal end of the rod 138. The normal bias from the spring 140 on the rod 138 biases the shoulder 146 against the contact arm 144, and causes the trigger 142 to assume the normal position shown in FIG. 2, with a release arm 148 of the trigger 142 extending generally parallel with the elongated dimension of the bottom handle 26. To disengage the ratchet pawl 136 from the ratchet teeth 134, the trigger 142 is squeezed which causes the release arm 148 to pivot counterclockwise as shown in FIG. 6. The counterclockwise movement of the contact arm 144 against the shoulder 146 moves the rod 138 in the distal direction, as shown in FIG. 6, and the distal movement of the rod 136 releases the engagement of the ratchet pawl 136 with the ratchet teeth 134. With the ratchet pawl 136 released from the ratchet teeth 134, the handles 24 and 26 are free to move away from one another.

The power control device 48 responds to the temperature-related signals from the thermocouples 110 supplied on the conductors 120 and 122 to control the amount of current delivered through the conductors 106 in 108 to the heating elements 49. The power control device 48 may execute any feedback control algorithm to control the current and hence temperature of the jaws 34 and 36. One beneficial feedback control algorithm is a well-known proportional, integral, derivative (PID) computation which offers the advantage of predictive capability for controlling the temperature of the jaws. The power control device 48 responds to the force-related signals from the strain gauge 126 supplied on the conductors 130 to initiate the delivery of the electrical power impulse impulse and to continue delivering the electrical power impulse so long as sufficient force is maintained on the tissue compressed between the working surfaces 38 and 40 of the jaws.

The strength and integrity of the seals created by use of the present invention have been evaluated using burst tests. To evaluate the strength of the seal with a burst test, the lumen of the vessel is connected to a source of pressurized fluid, such as air, which inflates the vessel adjacent to the sealed area until a rupture or burst in the sealed area or the vessel wall occurs. The fluid pressure at the rupture point is measured, and the rupture pressure represents the strength of the seal. The test is repeated many times with different specimens of sealed tissue. A sufficient number of burst tests are conducted to obtain a statistically significant number of samples by which to evaluate the strength and integrity of the seals. The burst tests indicate that the seals formed have some range of variability in strength, and the seal strength is dependent upon the type and the size of the vessel sealed. Despite the variations in the seal strength, the burst pressures observed indicate that the seals formed have more than sufficient strength to reliably withstand the applicable physiological pressures, and in most cases, multiples of those pressures.

The following example illustrates the utility of the smooth working surfaces 38 and 40 in vessel fusion. In a six-hour laboratory experiment, seals were formed on 101 samples of in vivo porcine tissue that included arteries, veins, tissue bundles, and mesentery. The tissue fusion instrument used in the experiment had aluminum nitride jaws which had been hand polished to an Ra of about 0.15 microns as determined by at least five measurements over the working surfaces. The aluminum nitride ceramic jaws had a width dimension of 5 mm and a length dimension of 25 mm and a thickness dimension of 1.5 mm. One of the working surfaces was crowned (FIG. 12) and the other working surface was flat or planar (FIG. 11). Each sample of tissue was compressed between the jaw working surfaces with a force of 110 N-150 N, resulting in a squeezed tissue thickness of about 0.05-0.10 mm. An impulse of power having a power density of 1500 W/in² (233 W/cm²) was delivered by the power control device 48 (FIG. 1) to the heating elements in the jaws. The power impulse had a 2.0 second time duration. The impulse produced enough thermal energy to successfully seal the tissue in each of the 101 instances. The thermocouples of the jaws recorded peak temperatures of the jaw working surfaces of about 150-180° C. during the impulse.

After each seal was formed, the jaws were separated to release the fused tissue. The tissue was considered stuck to one of the working surfaces if the fused tissue did not separate itself immediately from the working surface which contacted the tissue. Of the 101 tissue samples fused in the experiment, none adhered to the working surfaces of the jaws using this sticking evaluation criteria. Moreover, on occasion, blood or other tissue was present on the working surfaces of the jaws before the tissue sample was compressed between the jaws. Even in these adverse situations, the tissue did not stick to the smooth working surfaces. The blood or other tissue initially present on the jaws adhered to the tissue sample fused, thereby producing clean working surfaces, but there was no adherence between the fused tissue and the smooth working surfaces. Use of the present invention subsequent to this experiment has also confirmed the non-stick performance of the polished working surfaces.

The smooth working surfaces have been discussed in detail for an electrothermal instrument which transfers thermal energy for sealing tissue or for simultaneously fusing and cutting tissue. Other types of electrothermal instruments are a cautery knife or blade which is coated with the ceramic, such as aluminum nitride, and then polished to a smoothness which reduces the adhesion of tissue to the working surface. Such cautery knives or blades are typically used to cut or sever tissue or to coagulate bleeding from the tissue The smooth nonstick working surfaces of the present invention can also be used with electrosurgical tissue fusion devices, which conduct electrical energy, typically radio frequency (RF) current, from bipolar electrodes through tissue which is gripped between the bipolar electrodes. Under such circumstances, the bipolar electrodes or jaws are formed from ceramic, such as aluminum nitride, which has been doped with a material that causes the ceramic to be electrically conductive, to enable the conduction of current between the jaws through the tissue. The doping material can comprise relatively small particles of conductive material, such as metal or carbon, which is immiscible within the ceramic. The concentration of the immiscible conductive particles provides a matrix within the ceramic through which the RF current is conducted.

Alternatively, the ceramic can be doped with a material that is miscible with the ceramic. In this method of doping, the electrical conductivity of the ceramic is increased through atomic or molecular substitutions within the ceramic surface microstructure or through particle inclusions within a matrix of the ceramic or ceramic-like material surface microstructure. As a result of the increased conductivity, RF current can be conducted through the ceramic to the tissue. Of course, the conductive jaws must be electrically isolated from the remaining portions of the fusion instrument, so that the electrical energy is delivered between the jaws and not short-circuited through the instrument.

The smooth working surfaces described herein allow electrothermal procedures to be conducted without the tissue sticking to the jaws or other elements which contact the tissue. The smooth working surfaces are particularly important when sealing or fusing tissue or simultaneously cutting and sealing tissue, since tissue sticking after sealing can result in destroying or compromising the strength of the fused interface. Vessels sealed with the smooth working surfaces should possess greater integrity and strength, thereby decreasing the likelihood that the seal will ultimately fail. Reliable vessel seals are created considerably faster than with the prior art tissue fusion techniques now commonly used. The vessel seals are significantly stronger and more reliable than the seals created using common prior art tissue fusion devices. Confining the energy to the sealed area without significantly spreading that energy to damage adjacent tissue avoids compromising the integrity of the sealed area due to weakened adjoining tissue.

The significance of these and many other improvements and advantages will become apparent upon gaining a full appreciation of the ramifications and improvements of the present invention. Preferred embodiments of the invention and many of its improvements have been described with a degree of particularity. The description is of preferred examples of implementing the invention, but the description is not necessarily intended to limit the scope of the invention. The scope of the invention is defined by the following claims.

The invention claimed:

1. One of an electrothermal or electrosurgical instrument having a working surface from which energy is applied to tissue during a surgical procedure and which, reduces sticking of tissue to the working surface during the procedure, wherein the working surface is formed of ceramic material and the working surface has a smoothness defined by an Ra of no greater than 0.40 microns.

2. An instrument as defined in claim 1, wherein the working surface has a smoothness defined by an Ra of no greater than 0.25 microns.

3. An instrument as defined in claim 1, wherein the working surface has a smoothness defined by an Ra of no greater than 0.20 microns.

4. An instrument as defined in claim 1, wherein the the working surface has a smoothness defined by an Ra of no greater than 0.15 microns.

5. An instrument as defined in claim 1, wherein the ceramic material is essentially aluminum nitride.

6. An instrument as defined in claim 5, wherein the aluminum nitride on the working surface has been polished to achieve the defined smoothness.

7. An instrument as defined in claim 1, wherein the ceramic material includes a heating element embedded therein.

8. An instrument as defined in claim 1, wherein the ceramic material transfers thermal energy to the tissue during the electrothermal procedure.

9. An instrument as defined in claim 1, wherein the ceramic material contains electrically conductive particles to conduct current from the working surface to the tissue.

10. An instrument as defined in claim 1, wherein the ceramic material transfers compression force to the tissue during the procedure.

11. An instrument as defined in claim 10, further comprising two jaws, arms to which the jaws are attached, and a movement mechanism which moves the arms and the attached jaws toward one another, each jaw having a working surface, the working surface of each jaw having a linear dimension, and the movement mechanism moves the working surfaces toward and away from one another during the procedure with the linear dimensions of the working surfaces parallel to one another.

12. An instrument as defined in claim 11, wherein each of the working surfaces is substantially planar.

13. An instrument as defined in claim 11, wherein at least one of the working surfaces as an outwardly convex curvature facing the other working surface.

14. An instrument as defined in claim 11, wherein an extremity of the working surface includes a rounded edge.

15. An instrument as defined in claim 1, wherein the ceramic material includes a temperature sensor connected thereto.

16. A method of reducing sticking of tissue to a working surface of one of an electrothermal or electrosurgical instrument after applying energy to tissue during one of a tissue fusion or a tissue fusion and simultaneous cutting procedure, wherein the working surface is formed of ceramic material and the working surface which has a smoothness defined by an Ra of no greater than 0.40 microns.

17. A method as defined in claim 16, wherein the ceramic material comprises aluminum nitride.

18. A method as defined in claim 16, further comprising using a working surface which has an Ra of no greater than 0.25 microns.

19. A method as defined in claim 16, further comprising using a working surface which has an Ra of no greater than 0.20 microns.

20. A method as defined in claim 16, further comprising using a working surface which has an Ra of no greater than 0.15 microns.

21. A method as defined in claim 16, further comprising polishing the ceramic material at working surface with an abrasive to achieve the defined smoothness before using the instrument in the procedure.

22. A method as defined in claim 16, further comprising transferring thermal energy from the working surface to the tissue during the procedure.

23. A method as defined in claim 16, further comprising transferring electrical energy from the working surface into the tissue during the procedure.

24. A method as defined in claim 16, further comprising compressing the working surface against the tissue during the procedure.
